(19) United States
(12) Patent Application Publication
(10) Pub. No.: US 2005/0053644 A1
(43) Pub. Date: Mar. 10, 2005

(54) Indented antimicrobial paper and a process for making and using the same

(76) Inventors: Anthony S. Salemi, Des Plaines, IL (US); Daniel J. Mulvey, Elk Grove Village, IL (US); Anthony C. Pappageorge, Long Grove, IL (US)

Correspondence Address: PATENTS+TMS;A Professional Corporation, 1914 North Milwaukee Avenue, Chicago, IL, 60647, US

(21) Appl. No.: 10/659,089

(22) Filed: Sep. 10, 2003

(51) Int. Cl. A61K 9/70

(52) U.S. Cl. 424/443

(57) ABSTRACT

An indented antimicrobial paper and a process for making and using the same which prevents bacteria and/or microbial growth and/or migration are provided. The indented antimicrobial paper may have a base layer and an antimicrobial layer. Further, the indented antimicrobial paper may have an indented texture which has alternating high points and low points. The indented antimicrobial paper may have channels between the alternating high points and low points to prevent liquid pooling. The indented antimicrobial paper may cushion items placed on and/or absorb forces applied to the indented antimicrobial paper.

### BACKGROUND OF THE INVENTION

The present invention generally relates to a paper. More specifically, the present invention relates to an indented antimicrobial paper. The paper may cover a contaminated surface and/or providing a sanitized surface which prevents bacteria and/or microbe growth and/or migration. The indented antimicrobial paper may have a base first layer, such as, for example, a paper layer, a polyethylene layer, an adhesive layer, a polyurethane layer and/or the like. Further, the paper may have an antimicrobial layer and/or coating which contains an antimicrobial material. Moreover, the antimicrobial paper may be indented to provide a texture which, for example, resists slippage, cushions for cutting, prevents liquid pooling and/or the like.

It is generally known to provide a preparation sheet (hereinafter “prep sheet”) on a contaminated surface, such as, for example, kitchen counters, food preparation stations and/or food cutting boards when preparing foods. The prep sheet separates the foods prepared on top of the prep sheet from the contaminated surface below the prep sheet. Traditionally, the prep sheet is constructed from a material, such as, for example, a wax paper, a butcher, and/or the like. In more recent times, other materials, such as, for example, plastics, have been used as a prep sheet. Further, the prep sheet is a flat surface which is not indented. A prep sheet may become slippery and unsafe when liquids are in contact with the prep sheet. A slippery prep sheet causes foods being prepared on top of the prep sheet to slip from the prep sheet onto the contaminated surface. Still further, a slippery prep sheet causes cutting devices being used on top of the prep sheet to slip from the prep sheet and cause injuries. Moreover, the liquids which moisten the prep sheet may weaken the prep sheet and allow contaminants to pass through the prep sheet to the foods being prepared on top of the prep sheet.

Moreover, the prep sheet is often a thin sheet which punctures easily when in use with, for example, a cutting device. If the prep sheet is punctured, contaminants from the surface below contact and/or contaminate the foods prepared on top of the prep sheet. Additionally, the prep sheet is often contaminated even before being placed on the contaminated surface due to improper handling and/or packaging prior to use. The contaminated prep sheet causes the foods placed on the prep sheet to become contaminated.

Furthermore, the prep sheet is used in the medical and/or the dental industries to provide a covering on, for example, an examination table, an utensil tray and/or the like. Additionally, the prep sheet is used in the packing industry to protect contents of a package from contamination from outside sources, such as, bacteria, mold, contaminants, pollution and/or the like. Further, the prep sheet is used in the pet care industry as, for example, a liner in animal cages and/or litter boxes. Moreover, these prep sheets utilized in each of the above-mentioned industries also have the deficiencies of easily allowing contaminants to pass through the prep sheets due to being punctured and/or moistened and/or becoming slippery and/or unsafe when liquids are in contact with the prep sheets.

A need, therefore, exists for an indented antimicrobial paper and a process for making and using the same which may prevent bacteria and/or microbe growth and/or migration. Further, a need exists for an indented antimicrobial paper and a process for making and using the same which may stop contamination from contaminated surfaces in places, such as, for example, kitchens, laboratories, warehouses, animal cages, storage rooms, hospital rooms, examination rooms and/or the like. Still further, a need exists for an indented antimicrobial paper and a process for making and using the same which may be slip resistant and/or may prevent slippage from the indented antimicrobial paper onto the contaminated surface. Moreover, a need exists for an indented antimicrobial paper and a process for making and using the same which may channel liquids from the indented antimicrobial paper and/or may prevent liquid pooling on the indented antimicrobial paper. Furthermore, a need exists for an indented antimicrobial paper and a process for making and using the same which may provide a cushioning to cutting devices and/or objects placed on the indented antimicrobial paper and/or a padding to, for example, contents in a packaging container.

### SUMMARY OF THE INVENTION

The indented antimicrobial paper may be a disposable paper constructed from multiple layers, such as, for example, base layers, antimicrobial layers, adhesive layers, water resistant layers and/or the like. Further, the multiple layers may be more than three layers, such as, for example, eleven layers. The multiple layers, may be constructed from a material, such as, for example, a paper, a plastic, a polyurethane, a polyethylene, a thermoplastic, a polyethylene containing an antimicrobial material, a coating containing an antimicrobial material and/or the like. Still further, the antimicrobial material, such as, for example, silver zeolite may be contained in at least one of the multiple layers which may prevent bacteria and/or microbe growth and/or migration.

Furthermore, the indented antimicrobial paper may be, for example, indented to provide a texture. Moreover, the texture may have, for example, alternating high points and low points which may provide characteristics of, such as, for example, resisting slippage and/or cushioning a force from a cutting device. The texture may provide channels between the high points and the low points to prevent liquid pooling on the indented antimicrobial paper. Further, the indented antimicrobial paper may be placed on a contaminated surface, such as, for example, a counter, a table, a tray, a base of a box, a mat and/or the like. Still further, the indented antimicrobial paper may be used to provide sterilized packaging containers for foods and/or sterilized cushioning materials for the packaging of contents in a packaging container to be shipped.

To this end, in an embodiment of the present invention, a paper for providing a sanitized surface is provided. The paper has a base defined by a length and a width wherein the base has a top side and a bottom side wherein the bottom side is opposite the top side and further wherein the base forms a plane. Further, the paper has an antimicrobial surface associated with the top side of the base wherein the antimicrobial surface covers the top side. Moreover, the paper has a plurality of depressions formed in the base wherein the plurality of depressions extend outside of the plane of the base.

In an embodiment, the plurality of depressions are uniform across the base.

In an embodiment, the plurality of depressions are continuous from side to side on the base.

In an embodiment, the paper has an antimicrobial layer associated with the bottom surface of the base.

In an embodiment, the paper has a paper layer associated with the top side of the base wherein the paper layer is located between the antimicrobial surface and the base.

In an embodiment, the paper has a water resistant layer associated with the top side of the base wherein the water resistant layer is located between the antimicrobial surface and the base.

In an embodiment, the paper has a water resistant layer associated with the bottom side of the base.

In an embodiment, the paper has a plurality of water resistant layers associated with the top side of the base wherein the plurality of water resistant layers is located between the base and the antimicrobial surface. Further, the paper has a paper layer associated with the top side of the base wherein the paper layer is located between the antimicrobial surface and the base.

In an embodiment, the paper has a water resistant layer associated with the top side of the base wherein the water resistant layer is located between the base and the antimicrobial surface. Further, the paper has a the antimicrobial surface. Further, the paper has a plurality of paper layers associated with the top side of the base wherein the plurality of paper layers is located between the antimicrobial surface and the base.

In an embodiment, the paper has a water resistant layer associated with the top side of the base wherein the water resistant layer is located between the antimicrobial surface and the base. Further, the paper has a paper layer associated with the top side of the base wherein the paper layer is located between the antimicrobial surface and the base.

In an embodiment, the paper has a water resistant layer associated with the antimicrobial surface.

In another embodiment of the present invention, a process for making a paper is provided. The process has the steps of providing a sheet having a first side and a second side wherein the second side is opposite the first side and applying an antimicrobial layer to the first side of the sheet. Further, the process has the step of forming an indentation in the sheet wherein the indentation is uniform across the sheet.

In an embodiment, the indentation is continuous along the sheet.

In an embodiment, the process has the step of applying a water resistant layer to the second side of the sheet.

In an embodiment, the process has the steps of applying a water resistant layer to the first side of the sheet and scoring the water resistant layer wherein the antimicrobial layer is associated with the water resistant layer.

In an embodiment, the process has the step of applying a plurality of water resistant layers to the first side of the sheet wherein the antimicrobial layer is associated with the plurality of water resistant layers.

In an embodiment, the process has the step of adhering a paper layer to the first side of the sheet wherein the antimicrobial layer is associated with the paper layer.

In an embodiment, the process has the steps of applying a plurality of water resistant layers to the first side of the sheet and adhering a plurality of paper layers to the plurality of paper layers wherein the antimicrobial layer is associated with the plurality of water resistant layers.

In an embodiment, the process has the step of applying a water resistant layer to the antimicrobial layer.

In an embodiment, the process has the step of applying an antimicrobial surface to the second side of the sheet.

In an embodiment, the process has the step of adhering a paper layer to the first side of the sheet wherein the antimicrobial layer is associated with the paper layer.

In an embodiment, the process has the step of shredding the sheet.

In an embodiment, the process has the steps of applying a water resistant layer to the first side of the sheet and scoring the water resistant layer. Further, the process has the step of adhering a paper layer to

In an embodiment, the process has the step of printing indicia onto the sheet.

In an embodiment, the process has the step of dividing the sheet into a plurality of sheets.

In another embodiment of the present invention, a method for using a paper to protect against contamination is provided. The method has the step of providing a sheet having a perimeter wherein the sheet has a bottom surface and a top surface wherein the top surface is opposite the bottom surface wherein an antimicrobial surface substantially covers the top surface and further wherein the sheet has an indented texture. Further, the method has the steps of positioning the sheet on a surface wherein the bottom surface of the sheet is adjacent to the surface and positioning an object on the antimicrobial surface wherein the object is within the perimeter of the sheet.

In an embodiment, the method has the step of wrapping the antimicrobial surface around the object.

In an embodiment, the method has the step of enclosing the object within the sheet wherein the object is surrounded by the antimicrobial surface.

In an embodiment, the method has the step of separating a liquid from the object on the antimicrobial surface wherein the liquid is associated with the indented texture of the sheet.

It is, therefore, an advantage of the present invention to provide an indented antimicrobial paper and a process for making and using the same which prevents bacteria and/or microbe growth and/or migration.

Another advantage of the present invention is to provide an indented antimicrobial paper and a process for making and using the same which is slip resistant.

And, another advantage of the present invention is to provide an indented antimicrobial paper and a process for making and using the same which grips foods and/or objects placed on top of the indented antimicrobial paper.

Yet another advantage of the present invention is to provide an indented antimicrobial paper and a process for making and using the same which prevents foods and/or objects placed on top of the indented antimicrobial paper from slipping off the indented antimicrobial paper onto a surface.

A further advantage of the present invention is to provide an indented antimicrobial paper and a process for making and using the same which cushions objects placed on top of the indented antimicrobial paper.

A still further advantage of the present invention is to provide an indented antimicrobial paper and a process for making and using the same which absorbs and/or cushions a force from a cutting device in contact with the indented antimicrobial paper.

Moreover, an advantage of the present invention is to provide an indented antimicrobial paper and a process for making and using the same which channels liquids off of the indented antimicrobial paper to prevent liquid pooling and/or slippage caused from liquid pooling.

And, another advantage of the present invention is to provide an indented antimicrobial paper and a process for making and using the same which prevents food slippage and surface contamination caused from liquid pooling.

Yet another advantage of the present invention is to provide an indented antimicrobial paper and a process for making and using the same which provides a sanitized surface to cover a contaminated surface.

Another advantage of the present invention is to provide an indented antimicrobial paper and a process for making and using the same which protects foods being prepared and/or objects placed on top of the indented antimicrobial paper from contaminants on a surface.

Yet another advantage of the present invention is to provide an indented antimicrobial paper and a process for making and using the same which provides a sanitized packaging for foods and/or objects to be shipped.

A still further advantage of the present invention is to provide an indented antimicrobial paper and a process for making and using the same which provides a sanitized wrapping for foods and/or objects to be transported.

Moreover, an advantage of the present invention is to provide an indented antimicrobial paper and a process for making and using the same which provides a sanitized liner for boxes, shelves, tables, pans, cages, floors, trays, carts, seats and/or the like.

And, another advantage of the present invention is to provide an indented antimicrobial paper and a process for making and using the same which provides a sanitized bib for individual use.

Yet another advantage of the present invention is to provide an indented antimicrobial paper and a process for making and using the same which provides a sanitized cover for examination tables and/or utensil trays.

A further advantage of the present invention is to provide an indented antimicrobial paper and a process for making and using the same which provides a polyethylene layer to resist water and/or liquids.

A still further advantage of the present invention is to provide an indented antimicrobial paper and a process for making and using the same which is heavy duty to be cut and/or puncture resistant.

Moreover, an advantage of the present invention is to provide an indented antimicrobial paper and a process for making and using the same which has print relating to a manufacturer on the indented antimicrobial paper.

And, another advantage of the present invention is to provide an indented antimicrobial paper and a process for making and using the same which provides a liner for shoes and/or slippers that prevent microbe growth and/or migration.

Yet another advantage of the present invention is to provide an indented antimicrobial paper and a process for making and using the same which provides a wall underlayment before sheet rock for use in the construction industry.

A further advantage of the present invention is to provide an indented antimicrobial paper and a process for making and using the same which provides an outer layer of wall insulation for use in the construction industry.

Additional features and advantages of the present invention are described in, and will be apparent from, the detailed description of the presently preferred embodiments and from the drawings.

### BRIEF DESCRIPTION OF THE DRAWINGS

FIG. 1 illustrates a perspective view of an indented antimicrobial paper in an embodiment of the present invention.

FIG. 2 illustrates a side view of an indented antimicrobial paper in another embodiment of the present invention.

FIG. 3 illustrates a side view of an indented antimicrobial paper in another embodiment of the present invention.

FIG. 4 illustrates a side view of an indented antimicrobial paper in another embodiment of the present invention.

FIG. 5 illustrates a side view of an indented antimicrobial paper in another embodiment of the present invention.

FIG. 6 illustrates a side view of an indented antimicrobial paper in another embodiment of the present invention.

FIG. 7 illustrates a side view of an indented antimicrobial paper in another embodiment of the present invention.

FIG. 8 illustrates a side view of an indented antimicrobial paper in another embodiment of the present invention.

FIG. 9 illustrates a side view of an indented antimicrobial paper in another embodiment of the present invention.

FIG. 10 illustrates a side view of an indented antimicrobial paper in another embodiment of the present invention.

FIG. 11a illustrates a flowchart of a process of making the indented antimicrobial paper in FIG. 1 in an embodiment of the present invention.

FIG. 11b illustrates a flowchart of a process of making the indented antimicrobial paper in FIG. 1 in an embodiment of the present invention.

FIG. 12 illustrates a flowchart of a process of making the indented antimicrobial paper in FIG. 2 in an embodiment of the present invention.

FIG. 13 illustrates a flowchart of a process of making the indented antimicrobial paper in FIG. 3 in an embodiment of the present invention.

FIG. 14 illustrates a flowchart of a process of making the indented antimicrobial paper in FIG. 4 in an embodiment of the present invention.

FIG. 15 illustrates a flowchart of a process of making the indented antimicrobial paper in FIG. 5 in an embodiment of the present invention.

FIG. 16 illustrates a flowchart of a process of making the indented antimicrobial paper in FIG. 6 in an embodiment of the present invention.

FIG. 17 illustrates a flowchart of a process of making the indented antimicrobial paper in FIG. 7 in an embodiment of the present invention.

FIG. 18 illustrates a flowchart of a process of making the indented antimicrobial paper in FIG. 10 in an embodiment of the present invention.

FIG. 19 illustrates a flowchart of a process of making the indented antimicrobial paper in FIG. 8 in an embodiment of the present invention.

FIG. 20 illustrates a flowchart of a process of making the indented antimicrobial paper in FIG. 9 in an embodiment of the present invention.

### DETAILED DESCRIPTION OF THE PRESENTLY PREFERRED EMBODIMENTS

The present invention generally relates to a paper. More specifically, the present invention relates to an indented antimicrobial paper which may be used to cover and/or line a surface to provide a sanitized surface and/or prevent bacteria and/or microbe growth and/or migration. It should be understood that the indented antimicrobial paper may be suitable for use as cover and/or liner of a surface which require sanitary conditions, such as, for example, a medical utensil tray, an infant scale, a food prep table and/or the like. Additionally, the uses of the indented antimicrobial paper described hereinafter should not be construed as limited to the uses set forth for any one or more of the papers. The indented antimicrobial papers may have a base layer, such as, for example, a paper layer, a cardboard and/or the like. Further, the indented antimicrobial paper may have an antimicrobial layer and/or a coating which may have an antimicrobial material. Moreover, a surface of the indented antimicrobial paper may be pressed forming an indented texture. The texture of the surface may, for example, resist slippage; cushion items, prevent liquid pooling and/or the like. More than one base layer may be fused together when the indented texture is pressed onto the surface of the indented antimicrobial paper.

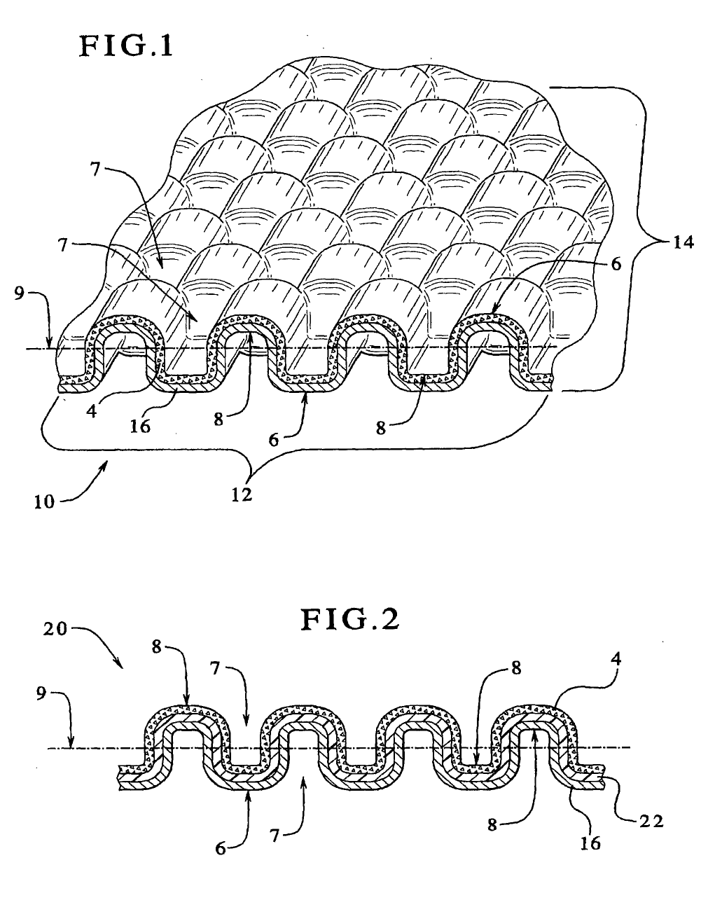

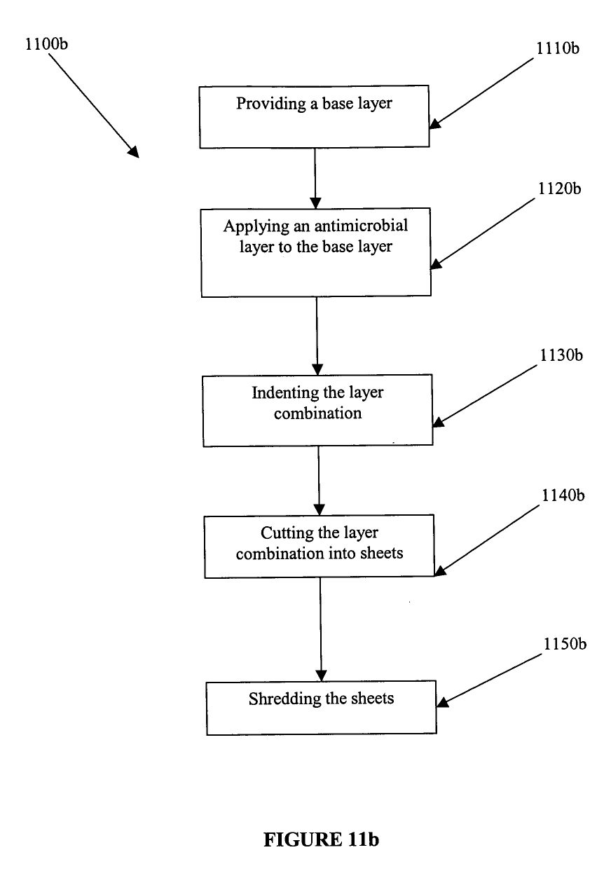

Referring now to the drawings wherein like numerals refer to like parts, FIG. 1 illustrates an indented antimicrobial paper 10 in an embodiment of the present invention. Additionally, FIG. 11 illustrates a flowchart 1100 for a process of making the indented antimicrobial paper 10 as illustrated in FIG. 1 in an embodiment of the present invention. The indented antimicrobial paper 10 may be placed on a surface, such as, for example, a base of a box, a shelf, a table, a pan, a cage, a floor, a tray, a cart, a seat and/or the like. The surface may contain contaminants, such as, for example, bacteria, microbes, fungus, mold spores, pollution, pollen and/or the like. Further, the indented antimicrobial paper 10 may be suitable for use in, for example, a kitchen, a laboratory, a warehouse, an animal cage, a storage room, a hospital room, an examination room and/or the like.

The indented antimicrobial paper 10 may have a length 12 and a width 14. Still further, the length 12 and the width 14 may be any size, such as, for example, twelve inches by eighteen inches, twenty-four inches by twenty-four inches, thirty-six inches by twenty-four inches and/or the like. It should be understood that the shape of the indent antimicrobial paper 10 may be any shape, such as, for example, a square, a rectangle, a circle, an oval, a triangle, an eclipse, a hexagon and/or the like.

Moreover, the indented antimicrobial paper 10 may have an antimicrobial layer 4 which may be applied to a base layer 16. The base layer 16 may be made from a material, such as, for example, a paper, a cardboard and/or the like. The paper may be, for example, a recycled kraft paper, a virgin kraft paper, a grey bogus paper, a news print paper, a butcher paper, a bleached kraft paper and/or the like. Further, the paper may be in a roll having a length, such as, for example, one foot, thirty inches, five foot and/or the like. The paper of the base layer 16 may have a weight range between, for example, 16.5 pounds and 90.00 pounds. Moreover, the antimicrobial layer 4 may prevent, for example, bacteria and/or microbe growth and/or migration. The antimicrobial layer 4 may contain an active antimicrobial ingredient, such as, for example, silver zeolite. The antimicrobial layer 4 may be a coating solution containing the active antimicrobial ingredient. Further, the antimicrobial layer 4 may be a substrate, such as, for example, polyethylene containing the active antimicrobial ingredient. Moreover, the antimicrobial layer 4 may be, for example, a polyethylene covered with the antimicrobial coating solution.

Furthermore, indicia 18 may be applied to the base layer 16 of the indented antimicrobial paper 10. The indicia 18 may relate to, for example, an entity, such as, for example, a manufacturer of the indented antimicrobial paper 10. The indicia 18 may be applied to the base layer 16 by, for example, a web printing press, a screening technique, a computer printer, a laser printer and/or the like.

As further illustrated in FIG. 1, the indented antimicrobial paper 10 may be textured having high points 6 and low points 8. The high points 6 and low points 8 may have a plane 9 which contains the midpoints between the high points 6 and the low points 8. Further, the high points 6 and the low points 8 may alternate across the length 12 and/or the width 14 with respect to the plane 9. The high points 6 and/or low points 8 may be depressions having shapes, such as, for example, a square, a triangle, a circle, a rectangle, a pentagon, an octagon and/or the like. Still further, the high points 6 and/or the low points 8 may form, for example, ribs (not shown in the drawings) which extend along the length 12 and/or width 14 of the indented antimicrobial paper 10. The ribs may be configured in a formation, such as, for example, rows extending across the length 12. Moreover, the ribs may be diagonal with respect to the length 12 and/or width 14. It should be understood that the high points 6 and the low points 8 may be depressions having any shapes and/or configurations, such as, for example, ribs which form alternating rectangles with respect to the plane 9 on the indented antimicrobial paper 10.

The high points 6 and/or the low points 8 may prevent objects, such as, for example, foods from slipping from the indented antimicrobial paper 10 onto the surface. The alternating high points 6 and the low points 8 may create a cushioning for objects placed on the indented antimicrobial paper 10. Further, the high points 6 and the low points 8 may provide a cushioning which may absorb a force applied from an object, such as, for example, a cutting device and/or the like.

Still further, the channels 7 may be provided between the high points 6 and the low points 8 on the indented antimicrobial paper 10. The channels 7 may extend along the length 12 and/or width 14 of the indented antimicrobial paper 10. Still further, the channels 7 may prevent liquid pooling on the indented antimicrobial paper 10. Moreover, the high points 6 and the low points 8 may force liquids into the channels 7 and/or move the liquids through the channels 7 from the indented antimicrobial paper 10 onto the surface.

Furthermore, the indented antimicrobial paper 10 may be suitable for use as a liner in, for example, shelves, refrigerated food cases, packaging containers, animal cages, animal litter boxes and/or the like. Additionally, the indented antimicrobial paper 10 may be suitable for use as a cover on, for example, counters, food serving surfaces and/or the like. In an embodiment of the indented antimicrobial paper 10, the base layer 16 may be made from a material, such as, for example, an examination table cover paper and may be suitable for use as a table cover, a utensil trays, a liner for food trays, a dental bib and/or the like. In another embodiment of the indented antimicrobial paper 10, the base layer 16 may be made from a paper, such as, for example, kraft paper and may be suitable for use as a liner for shoes and/or slippers. In yet another embodiment of the indented antimicrobial paper 10, the base layer 16 may be made from a paper, such as, for example, bleached kraft paper and may be suitable for use as a wall underlayment before sheet rock and/or an outer layer of wall insulation. Moreover, the indented antimicrobial paper 10 may be used as, for example, a liner, a bedding, a cover, a mat, a bib, a wrap, a package and/or the like.

Referring to Figure 11a, a flowchart 1100 of a process of making the indented antimicrobial paper 10 is provided. First, a base layer 16 may be provided via step 1110. Further, the indicia 18 may be printed on the base layer 16 with, for example, the web printing press. An antimicrobial layer 4 may be applied to the base layer 16 with a coating applicator and/or an extrusion applicator via step 1120. Still further, the layer combination via step 1120 which may be pressed with, for example, an indentor to texture the layer combination with the high points 6 and low points 8 and/or channels 7 via step 1130. Moreover, the layer combination of step 1130 may be cut into sheets for packaging with, for example, a sheeter via step 1140.

FIG. 11b further illustrates the flow chart 1100. The sheets in step 1140 of FIG. 11a may be shredded into, for example, long narrow strips with, for example, a paper shredder via step 1150. Moreover, the long narrow strips of step 1150 may be used as, for example, an animal bedding, a packaging container filler and/or the like.

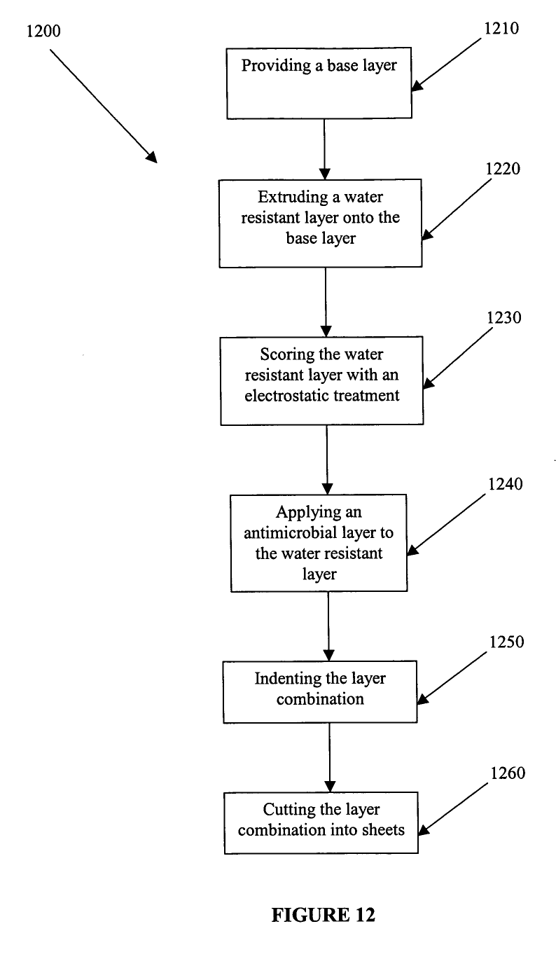

FIG. 2 and FIG. 12 illustrate an indented antimicrobial paper 20 and a flowchart 1200 for a process of making the indented antimicrobial paper 20 in embodiments of the present invention, respectively. The indented antimicrobial paper 20 may have the length 12 and the width 14 and/or may be placed upon the surface. The indicia 18 may be applied to the base layer 16. Further, the indented antimicrobial paper 20 may have a water resistant layer 22 applied to the base layer 16. The water resistant layer 22 may be made from a material, such as, for example, a polyethylene, a thermoplastic, a plastic, a polyurethane, and/or the like. Still further, the water resistant layer 22 may be applied to the base layer 16 by, for example, an extrusion applicator.

As further illustrated in FIG. 2, the indented antimicrobial paper 20 may have the antimicrobial layer 4 attached to the water resistant layer 22. Moreover, the indented antimicrobial paper 20 may be textured with the high points 6 and the low points 8 and/or the channels 7. The indented antimicrobial paper 20 may be suitable for use as a liner in, for example, shelves, packaging containers, animal cages, litter boxes, floors surfaces, animal counter tops, refrigerated food cases, food sheet pans and/or the like. Further, the indented antimicrobial paper 20 may be suitable for use as a cover on, for example, food service surfaces, cutting boards, tables, floors, kitchen work stations and/or the like. Still further, the indented antimicrobial paper 20 may be suitable for use as, for example, a food wrap, a counter top, a food thawing mat and/or the like. Moreover, the indented antimicrobial paper 20 may be used as, for example, a liner, a bedding, a cover, a mat, a bib, a wrap, a package and/or the like.

Referring to FIG. 12, the flowchart 1200 may provide a base layer 16 via step 1210. Further, the indicia 18 may be printed on the base layer 16. A water resistant layer 22 may be, for example, extruded onto the base layer 16 with an extrusion applicator via step 1220. The water resistant layer 22 may be scored by, for example, an electrostatic treatment via step 1230. Still further, the antimicrobial layer 4 may be applied to the water resistant layer 22 with a coating applicator and/or an extrusion applicator via step 1240. Moreover, the layer combination of step 1240 may be pressed with, for example, an indentor to texture the layer combination with the high points 6 and the low points 8 and the channels 7 via step 1250. Furthermore, the layer combination of step 1250 may be cut into sheets for packaging with, for example, a sheeter via step 1260.

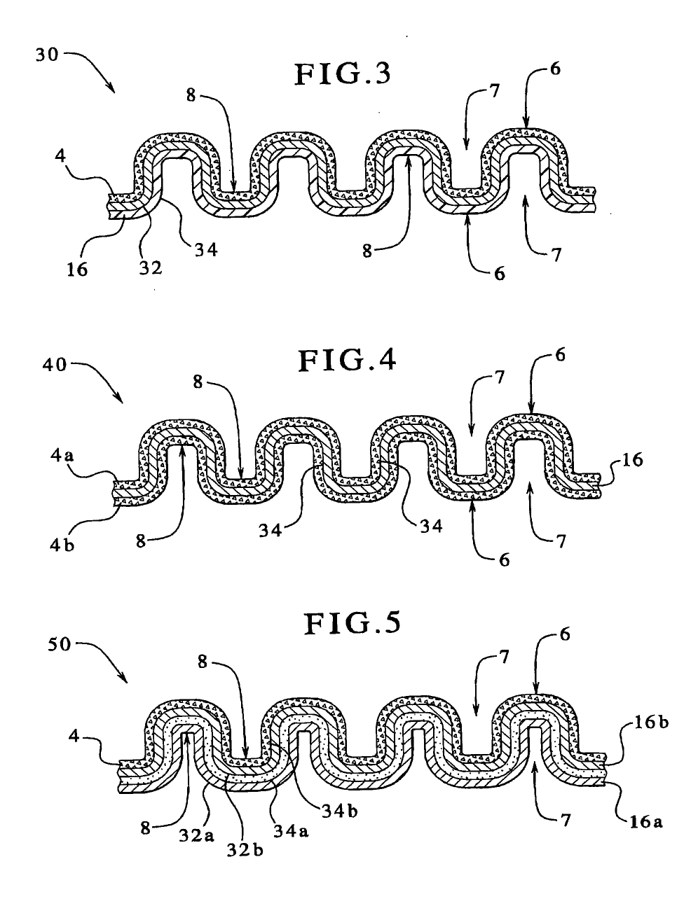

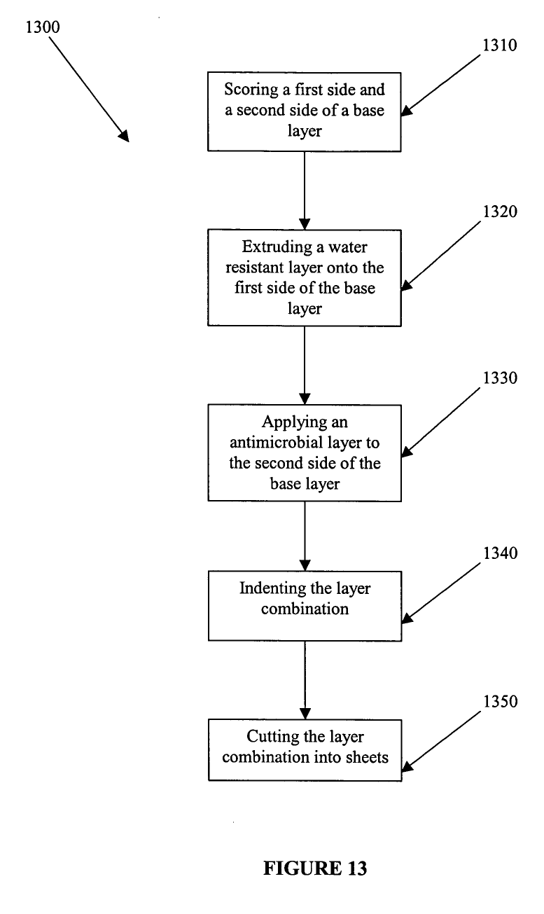

FIG. 3 and FIG. 13 illustrate an indented antimicrobial paper 30 and a flowchart 1300 of a process for making the indented antimicrobial paper 30 in embodiments of the present invention, respectively. The indented antimicrobial paper 30 may have the length 12 and the width 14 and/or may be placed upon the surface. The indicia 18 may be applied to the base layer 16. The base layer 16 may have a first side 32 and a second side 34 opposite the first side 32. Further, the indented antimicrobial paper 30 may have the water resistant layer 22 attached to the first side 32 of the base layer 16. Still further, the indented antimicrobial paper 30 may have the antimicrobial layer 4 attached to the second side 34 of the base layer 16.

Moreover, the indented antimicrobial paper 30 may be textured with the high points 6 and the low points 8 and/or the channels 7. The indented antimicrobial paper 30 may be suitable for use as a liner in, for example, trays, utensil carts, animal cages, infant scales, litter boxes, packaging containers and/or the like. Further, the indented antimicrobial paper 30 may be suitable for use as, for example, a dental bib, a seat cover, a floor mat, an animal food and/or water bowl mat and/or the like. Moreover, the indented antimicrobial paper 30 may be used as, for example, a bedding, a wrap, a package and/or the like.

Referring to FIG. 13, the flowchart 1300 may provide the first side 32 and the second side 34 of the base layer 16 via step 1310. Further, the indicia 18 may be printed on the base layer 16. The water resistant layer 22 may be, for example, extruded onto the first side 32 of the base layer 16 with an extrusion applicator via step 1320. Still further, the antimicrobial layer 4 may be applied to the second side 34 of the base layer 16 with a coating applicator and/or an extrusion applicator via step 1330. Moreover, the layer combination of 1330 may be pressed with, for example, an indentor to texture the layer combination with the high points 6 and low points 8 and/or the channels 7 via step 1340. Furthermore, the layer combination of step 1340 may be cut into sheets for packaging with, for example, a sheeter via step 1260.

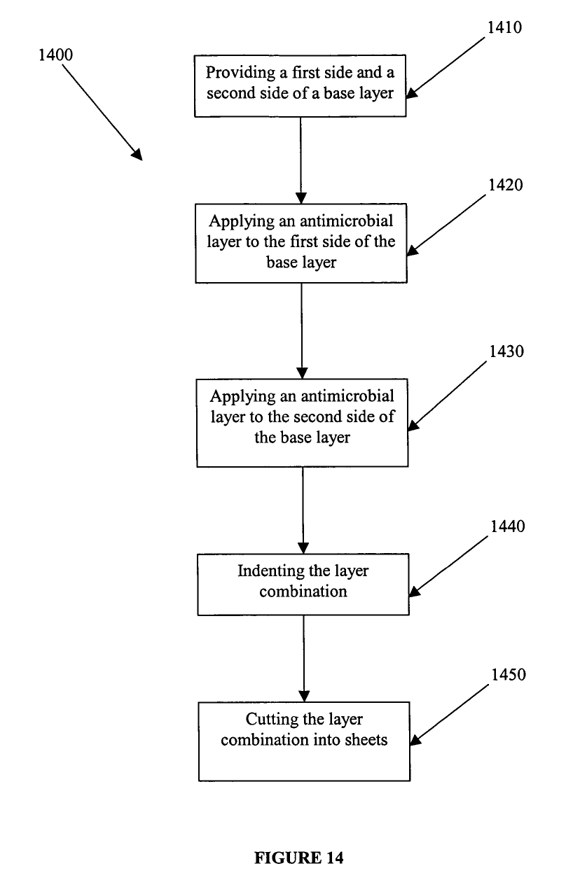

FIG. 4 and FIG. 14 illustrate an indented antimicrobial paper 40 and a flowchart 1400 for a process of making the indented antimicrobial paper 40 in embodiments of the present invention, respectively. The indented antimicrobial paper 40 may have the length 12 and the width 14 and/or may be placed upon the surface. The indicia 18 may be applied to the base layer 16. The base layer 16 may have the first side 32 and the second side 34. Further, the indented antimicrobial paper 40 may have the first antimicrobial layer 4a attached to the first side 32 of the base layer 16. Still further, the indented antimicrobial paper 40 may have the second antimicrobial layer 4b attached to the second side 34 of the base layer 16.

As further illustrated in FIG. 4, the indented antimicrobial paper 30 may be textured with the high points 6 and the low points 8 and/or the channels 7. Moreover, the indented antimicrobial paper 40 may be suitable for use as a liner in, for example, packaging containers, animal cages, litter boxes and/or the like. Furthermore, the indented antimicrobial paper 40 may be used as, for example, a bedding, a cover, a mat, a bib, a wrap, a package and/or the like.

FIG. 14 illustrates the flowchart 1400 which provides the first side 32 and the second side 34 of the base layer 16 via step 1410. The indicia 18 may be printed on the base layer 16. Further, the first antimicrobial layer 4a may be applied to the first side 32 of the base layer 16 with a coating applicator and/or an extrusion applicator via step 1420. Still further, the second antimicrobial layer 4b may be applied to the second side 34 of the base layer 16 with a coating applicator and/or an extrusion applicator via step 1430. Moreover, the layer combination of step 1430 may be pressed with, for example, an indentor to texture the layer combination with the high points 6 and the low points 8 and/or the channels 7 via step 1440. Furthermore, the layer combination of step 1440 may be cut into sheets for packaging with, for example, a sheeter via step 1260.

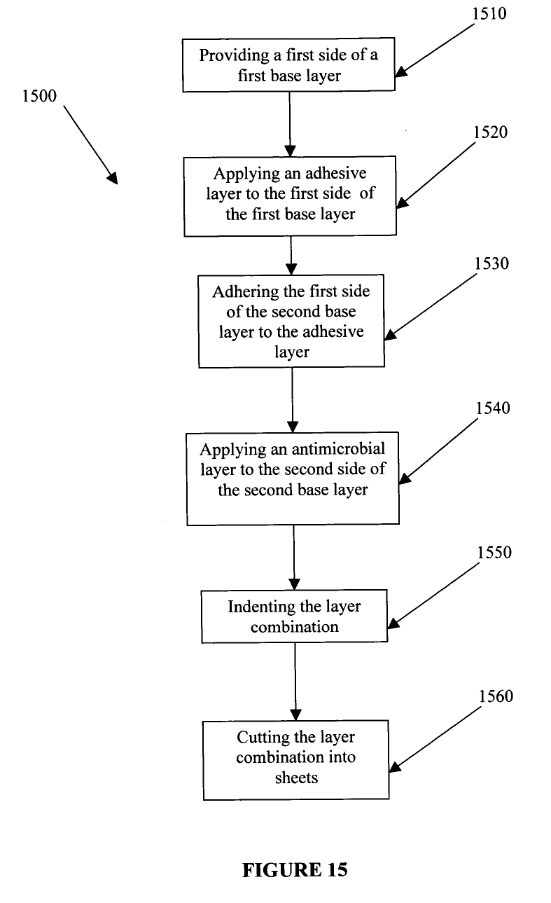

FIG. 5 and FIG. 15 illustrate an indented antimicrobial paper 50 and a flowchart 1500 for a process of making the indented antimicrobial paper 50 in embodiments of the present invention, respectively. The indented antimicrobial paper 50 may have the length 12 and the width 14 and/or may be placed on the surface. Further, the indented antimicrobial paper 50 may have an adhesive layer 52 connecting a first base layer 16a to a second base layer 16b. The adhesive layer 52 may be made of an adhesive substance, such as, for example, a glue.

Still further, the indicia 18 may be applied to the first base layer 16a and/or the second base layer 16b. The base layers 16a, 16b may have the first side 32a, 32b and the second side 34a, 34b, respectively. The indented antimicrobial paper 50 may have the antimicrobial layer 4 attached to the second side 34b of the second base layer 16b. Moreover, the indented antimicrobial paper 50 may be textured with the high points 6 and the low points 8 and/or the channels 7. Furthermore, the indented antimicrobial paper 50 may be suitable for use as a liner in, for example, packaging containers, animal cages, litter boxes and/or the like. Moreover, the indented antimicrobial paper 50 may be used as, for example, a bedding, a cover, a mat, a bib, a wrap, a package and/or the like.

FIG. 15 illustrates the flowchart 1500 which may provide the first base layer 16a via step 1510. The indicia 18 may be printed on the base layers 16a, 16b. An adhesive layer 52 may be applied to the first base layer 16a with, for example, an adhesive applicator via step 1520. The first side 32b of the second base layer 16b may be adhered to the first base layer 16a with, for example, glue from the adhesive applicator via 1530. Further, the antimicrobial layer 4 may be applied to the second side 32b of the second base layer 16b with a coating applicator and/or an extrusion applicator via step 1540. Still further, the layer combination of step 1540 may be pressed with, for example, an indentor to texture the layer combination with the high points 6 and low points 8 and/or the channels 7 via step 1550. Furthermore, the layer combination of step 1550 may be cut into sheets for packaging with, for example, a sheeter via step 1560.

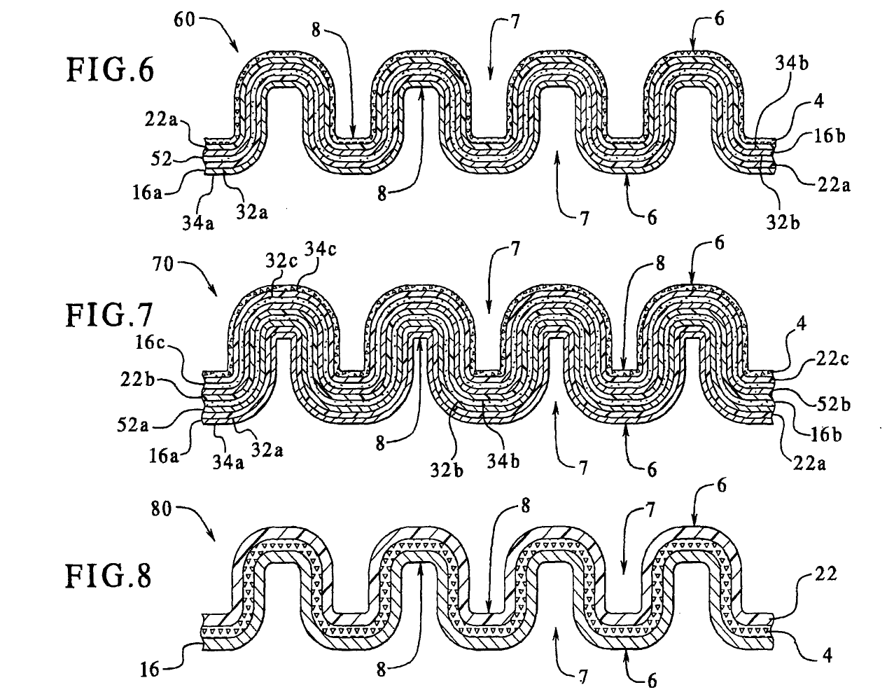

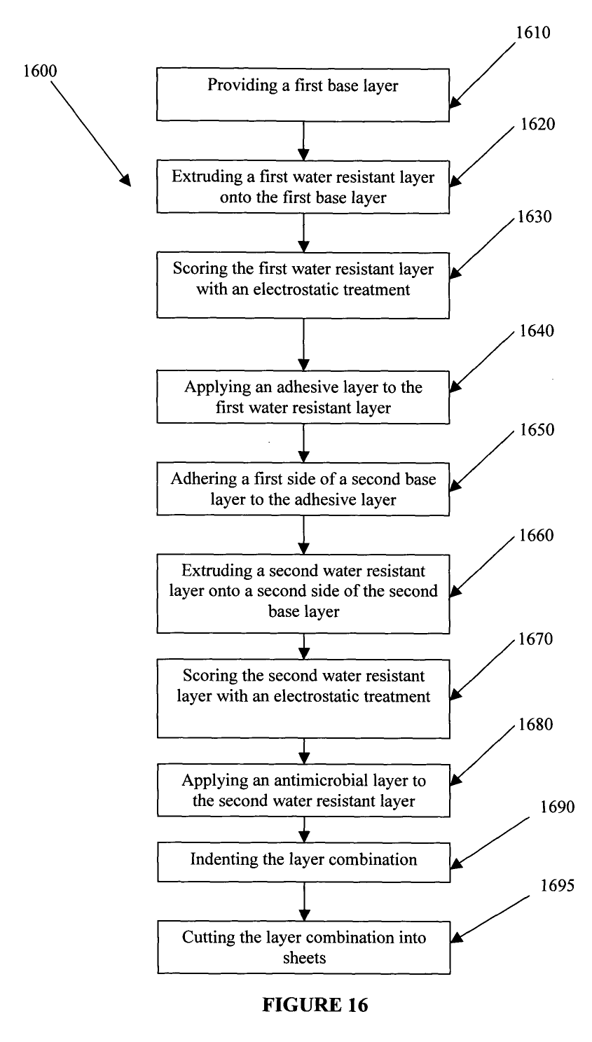

FIG. 6 and FIG. 16 illustrate an indented antimicrobial paper 60 and a flowchart 1600 for a process of making the indented antimicrobial paper 60 in embodiments of the present invention, respectively. The indented antimicrobial paper 60 may have the length 12 and the width 14 and/or may be placed on the surface. Further, the indented antimicrobial paper 60 may have an adhesive layer 52 connecting a first base layer 16a having first water resistant layer 22a to a second base layer 16b having a second water resistant layer 22b.

Still further, the indicia 18 may be applied to the first base layer 16a and/or second base layer 16b. The base layers 16a, 16b may have the first side 32a, 32b and the second side 34a, 34b, respectively. The indented antimicrobial paper 60 may have the antimicrobial layer 4 attached to the second water resistant layer 22b. Moreover, the indented antimicrobial paper 60 may be textured with the high points 6 and the low points 8 and/or the channels 7. Furthermore, the indented antimicrobial paper 60 may be suitable for use as a heavy duty surface, such as, for example, a food cutting board, a medical and/or animal laboratory cutting surface, a pathology table cover and/or the like. Moreover, the indented antimicrobial paper 60 may be used as, for example, a liner, a bedding, a cover, a mat, a bib, a wrap, a package and/or the like.

FIG. 16 illustrates the flowchart 1600 which may provide the first base layer 16a via step 1610. The first water resistant layer 22a may be, for example, extruded onto the first base layer 16a with an extrusion applicator via step 1620. Further, the first water resistant layer 22a may be scored by, for example, an electrostatic treatment via step 1630. The adhesive layer 52 may be applied to the first water resistant layer 22a with, for example, an adhesive applicator via step 1640. The first side 32b of the second base layer 16b may be adhered to the adhesive layer 52 with, for example, glue from the adhesive applicator via 1650. The indicia 18 may be printed on the first base layer 16a and/or the second base layer 16b with, for example, a web printing press. Still further, the second water resistant layer 22b may be, for example, extruded onto the second side 34b of the second base layer 16b with an extrusion applicator via step 1660. The second water resistant layer 22a may be scored by, for example, an electrostatic treatment via step 1670. Moreover, the antimicrobial layer 4 may be applied to the second water resistant layer 22b with a coating applicator and/or an extrusion applicator via step 1680. Still further, the layer combination of step 1680 may be pressed with, for example, an indentor to texture the layer combination with the high points 6 and the low points 8 and/or the channels 7 via step 1690. Furthermore, the layer combination of step 1690 may be cut into sheets for packaging with, for example, a sheeter via step 1695.

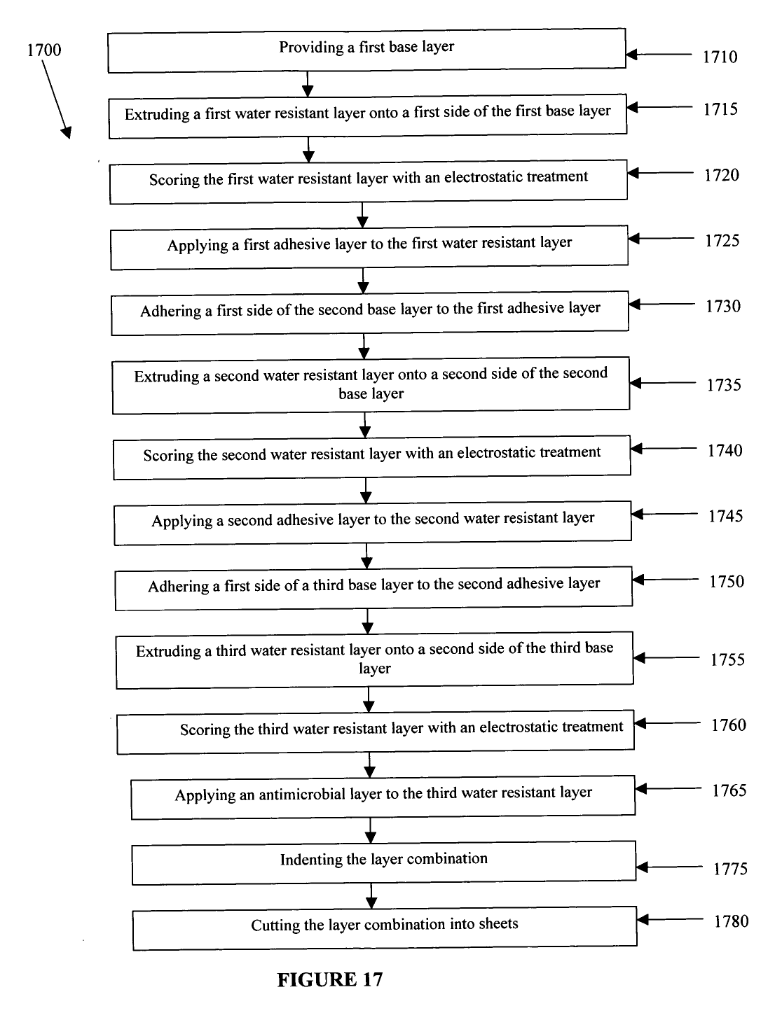

FIG. 7 and FIG. 17 illustrate an indented antimicrobial paper 70 and a flowchart 1700 for a process of making the indented antimicrobial paper 70 in embodiments of the present invention, respectively. The indented antimicrobial paper 70 may have the length 12 and the width 14 and/or may be placed on the surface. Further, the indented antimicrobial paper 70 may have a first adhesive layer 52a connecting a first base layer 16a having first water resistant layer 22a to a second base layer 16b having a second water resistant layer 22b. Still further, the indented antimicrobial paper 70 may have a second adhesive layer 52b connecting the second water resistant layer 22b to a third base layer 16c having a third water resistant layer 22c.

Still further, the indicia 18 may be applied to the first base layer 16a, the second base layer 16a and/or third base layer 16c. The base layers 16a, 16b, 16c may have the first sides 32a, 32b, 32c and the second sides 34a, 34b, 34c, respectively. The indented antimicrobial paper 70 may have the antimicrobial layer 4 attached to the third water resistant layer 22c. Moreover, the indented antimicrobial paper 70 may be textured with the high points 6 and the low points 8 and/or the channels 7. Furthermore, the indented antimicrobial paper 70 may be suitable for use as a heavy duty surface, such as, for example, a food cutting board, a medical and/or animal laboratory cutting surface, a pathology table cover and/or the like. Moreover, the indented antimicrobial paper 70 may be used as, for example, a liner, a bedding, a cover, a mat, a bib, a wrap, a package and/or the like.

FIG. 17 illustrates the flowchart 1700 which may provide the first base layer 16a via step 1710. The first water resistant layer 22a may be, for example, extruded onto the first base layer 16a with an extrusion applicator via step 1715. Further, the first water resistant layer 22a may be scored by, for example, an electrostatic treatment via step 1720. The first adhesive layer 52a may be applied to the first water resistant layer 22a with, for example, an adhesive applicator via step 1725. The first side 32b of the second base layer 16b may be adhered to the first adhesive layer 52a with, for example, glue from the adhesive applicator via 1730.

Still further, the second water resistant layer 22b may be, for example, extruded onto the second side 34b of the second base layer 16b with an extrusion applicator via step 1735. The second water resistant layer 22b may be scored by, for example, an electrostatic treatment via step 1740. Moreover, the second adhesive layer 52b may be applied to the second water resistant layer 22b with, for example, an adhesive applicator via step 1745. The first side 32c of the third base layer 16c may be adhered to the second adhesive layer 52b with, for example, glue from the adhesive applicator via 1750.

Furthermore, the third water resistant layer 22c may be, for example, extruded onto the second side 34b of the third base layer 16c with an extrusion applicator via step 1755. The third water resistant layer 22c may be scored by, for example, an electrostatic treatment via step 1760. The antimicrobial layer 4 may be applied to the third water resistant layer 22c with a coating applicator and/or an extrusion applicator via step 1765. Moreover, the layer combination of step 1765 may be pressed with, for example, an indentor to texture the layer combination with the alternating high points 6 and low points 8 and/or the channels 7 via step 1770. Further, the layer combination of step 1770 may be cut into sheets for packaging with, for example, a sheeter via step 1775.

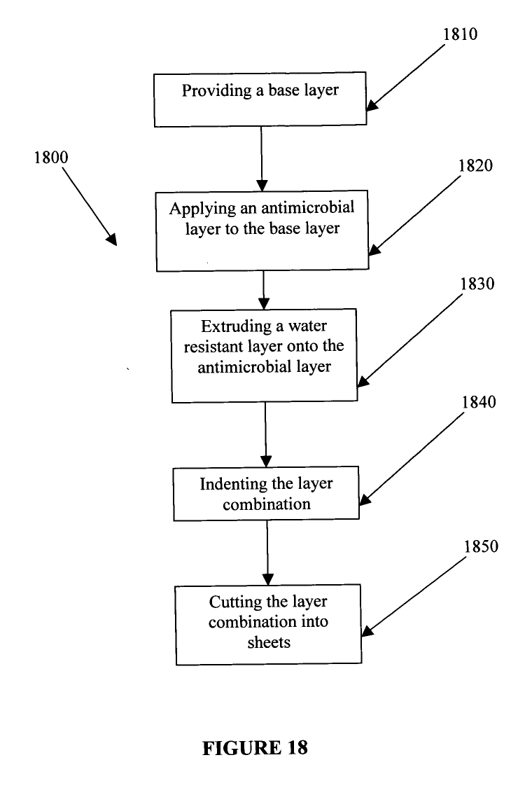

FIG. 8 and FIG. 18 illustrate an indented antimicrobial paper 80 and a flowchart 1800 for a process of making the indented antimicrobial paper 80 in embodiments of the present invention, respectively. The indented antimicrobial paper 80 may have the length 12 and the width 14 and/or may be placed on the surface. The indicia 18 may be applied to the base layer 16. Further, the indented antimicrobial paper 80 may have the antimicrobial layer 4 applied to the base layer 16. Still further, the water resistant layer 22 may be applied to the antimicrobial layer 4. Moreover, the indented antimicrobial paper 80 may be textured with the alternating high points 6 and the low points 8 and/or the channels 7. Furthermore, the indented antimicrobial paper 80 may be used as, for example, a liner, a bedding, a cover, a mat, a bib, a wrap, a package and/or the like.

Referring to FIG. 18, the flowchart 1800 may provide a base layer 16 via step 1810. Further, the indicia 18 may be printed on the base layer 16. The antimicrobial layer 4 may be applied to the base layer 16 with a coating applicator and/or an extrusion applicator via step 1820. Still further, the water resistant layer 22 may be, for example, extruded onto the antimicrobial layer 4 with an extrusion applicator via step 1830. Moreover, the layer combination of step 1830 may be pressed with, for example, an indentor to texture the layer combination with the high points 6 and the low points 8 and/or the channels 7 via step 1840. Furthermore, the layer combination of step 1840 may be cut into sheets for packaging with, for example, a sheeter via step 1850.

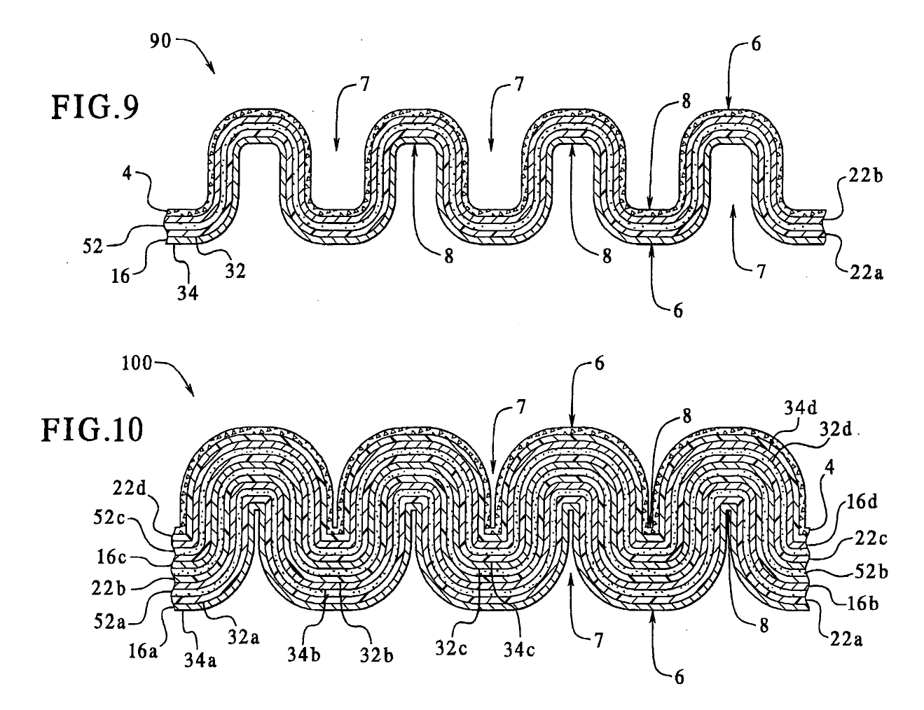

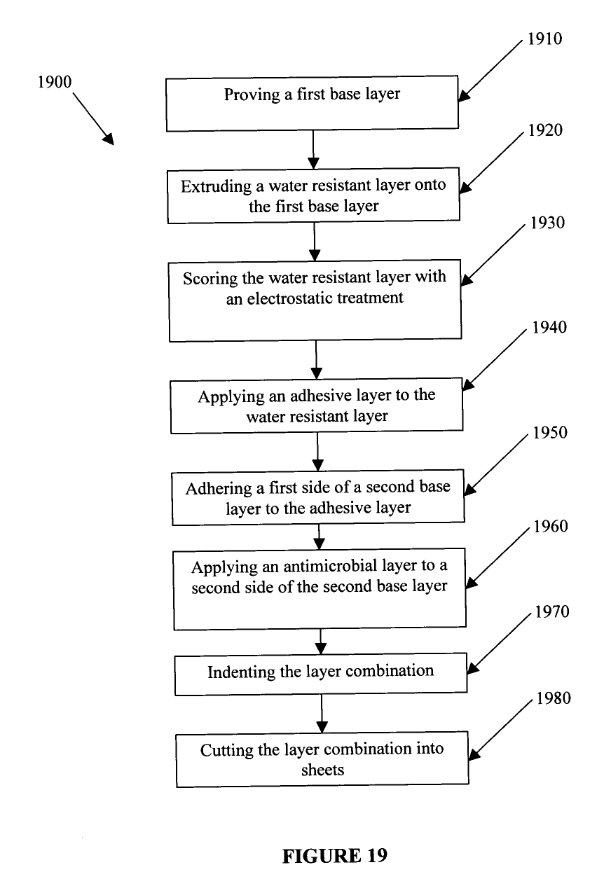

FIG. 9 and FIG. 19 illustrate an indented antimicrobial paper 90 and a flowchart 1900 for a process of making the indented antimicrobial paper 90 in embodiments of the present invention, respectively. The indented antimicrobial paper 90 may have the length 12 and the width 14 and/or may be placed on the surface. Further, the indented antimicrobial paper 90 may have the adhesive layer 52 connecting a first base layer 16a having the water resistant layer 22 to a second base layer 16b having the antimicrobial layer 4. Still further, the indicia 18 may be applied to the first base layer 16a and/or second base layer 16b. The base layers 16a, 16b may have the first sides 32a, 32b and the second sides 34a, 34b, respectively. Moreover, the indented antimicrobial paper 90 may be textured with the high points 6 and the low points 8 and/or the channels 7. Furthermore, the indented antimicrobial paper 90 may be used as, for example, a liner, a bedding, a cover, a mat, a bib, a wrap, a package and/or the like.

FIG. 19 illustrates the flowchart 1900 which may provide the first base layer 16a via step 1910. The water resistant layer 22 may be, for example, extruded onto the first base layer 16 with an extrusion applicator via step 1920. Further, the water resistant layer 22 may be scored by, for example, an electrostatic treatment via step 1930. The adhesive layer 52 may be applied to the water resistant layer 22 with, for example, an adhesive applicator via step 1940. The first side 32 of the second base layer 16b may be adhered to the adhesive layer 52 with, for example, glue from the adhesive applicator via 1950. The indicia 18 may be printed on the first base layer 16a and/or second base layer 16b with, for example, a web printing press. Still further, the antimicrobial layer 4 may be applied to the second side 34b of the second base layer 16b with a coating applicator and/or an extrusion applicator via step 1960. Moreover, the layer combination of step 1960 may be pressed with, for example, an indentor to texture the layer combination with the high points 6 and the low points 8 and/or the channels 7 via step 1970. Furthermore, the layer combination of step 1970 may be cut into sheets for packaging with, for example, a sheeter via step 1980.

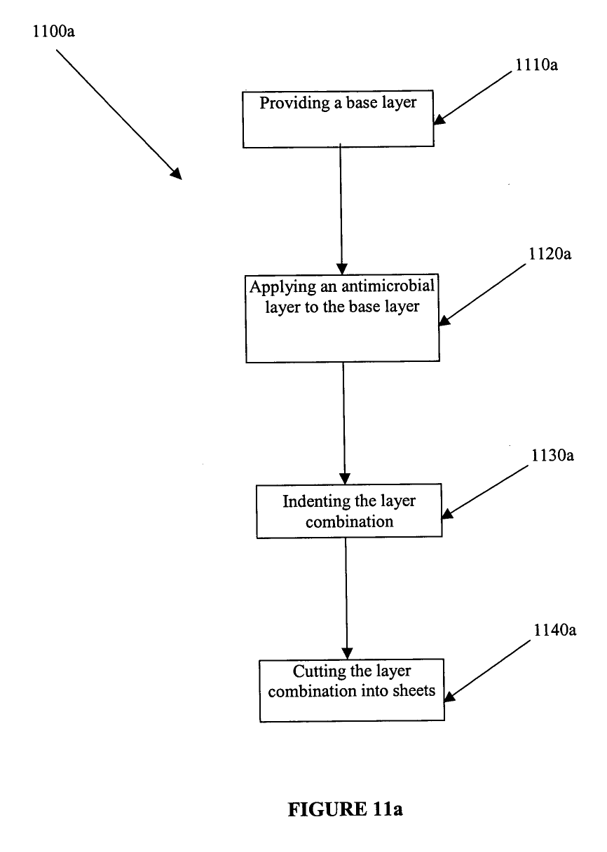

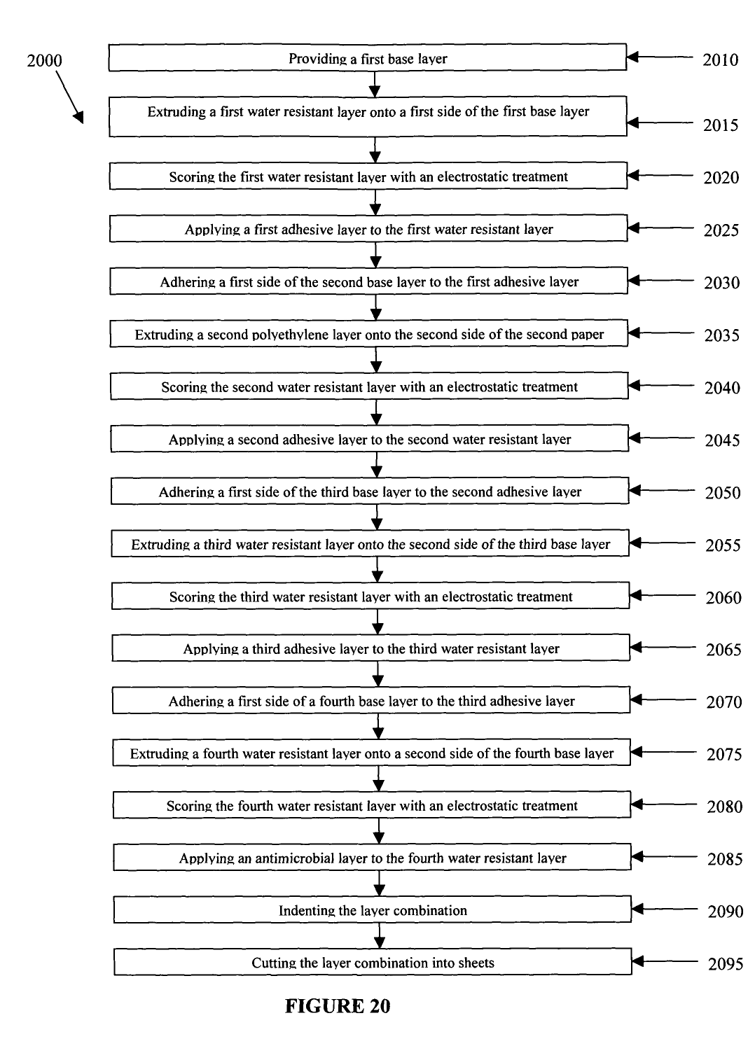

FIG. 10 and FIG. 20 illustrate an indented antimicrobial paper 100 and a flowchart 2000 for a process of making the indented antimicrobial paper 100 in embodiments of the present invention, respectively. The indented antimicrobial paper 100 may have the length 12 and the width 14 and may be placed on the surface. Further, the indented antimicrobial paper 100 may have a first adhesive layer 52a connecting a first base layer 16a having first water resistant layer 22a to a second base layer 16b having a second water resistant layer 22b. Still further, the indented antimicrobial paper 100 may have a second adhesive layer 52b connecting the second water resistant layer 22b to a third base layer 16c having a third water resistant layer 22c. Moreover, the indented antimicrobial paper 100 may have a third adhesive layer 52c connecting the third base layer 16c to a fourth base layer 16d having a fourth water resistant layer 22d.

Furthermore, the indicia 18 may be applied to the first base layer 16a, the second base layer 16b, the third base layer 16c and/or the fourth base layer 16d. The base layers 16a, 16b, 16c, 16d may have the first sides 32a, 32b, 32c, 32d and the second sides 34a, 34b, 34c, 34d. The indented antimicrobial paper 100 may have the antimicrobial layer 4 attached to the fourth water resistant layer 22d. Moreover, the indented antimicrobial paper 100 may be textured with the high points 6 and the low points 8 and/or the channels 7. Furthermore, the indented antimicrobial paper 100 may be used as, for example, a liner, a bedding, a cover, a mat, a bib, a wrap, a package and/or the like.

FIG. 20 illustrates the flowchart 2000 which may provide the first base layer 16a via step 2010. The first water resistant layer 22a may be, for example, extruded onto the first base layer 16a with an extrusion applicator via step 2015. Further, the first water resistant layer 22a may be scored by, for example, an electrostatic treatment via step 2020. The first adhesive layer 52a may be applied to the first water resistant layer 22a with, for example, an adhesive applicator via step 2025. The first side 32a of the second base layer 16a may be adhered to the first adhesive layer 52a with, for example, glue from the adhesive applicator via step 2030.

Still further, the second water resistant layer 22b may be, for example, extruded onto the second side 34b of the second base layer 16b with an extrusion applicator via step 2035. The second water resistant layer 22b may be scored by, for example, an electrostatic treatment via step 2040. Moreover, the second adhesive layer 52b may be applied to the second water resistant layer 22b with, for example, an adhesive applicator via step 2045. The first side 32c of the third base layer 16c may be adhered to the second adhesive layer 52b with, for example, glue from the adhesive applicator via step 2050.

Furthermore, the third water resistant layer 22c may be, for example, extruded onto the second side 34c of the third base layer 16c with an extrusion applicator via step 2055. The third water resistant layer 22c may be scored by, for example, an electrostatic treatment via step 2060. The third adhesive layer 52c may be applied to the third water resistant layer 22c with, for example, an adhesive applicator via step 2065. Moreover, the first side 32d of the fourth base layer 16d may be adhered to the third adhesive layer 52c with, for example, glue from the adhesive applicator via step 2070. The fourth water resistant layer 22d may be, for example, extruded onto the second side 34d of the forth base layer 16d with an extrusion applicator via step 2075.

Further, the fourth water resistant layer 22d may be scored by, for example, an electrostatic treatment via step 2080. The antimicrobial layer 4 may be applied to the fourth water resistant layer 22d with a coating applicator and/or an extrusion applicator via step 2085. Moreover, the layer combination of step 2085 may be pressed with, for example, an indentor to texture the layer combination with the high points 6 and the low points 8 and/or the channels 7 via step 2090. Further, the layer combination of step 2090 may be cut into sheets for packaging with, for example, a sheeter via step 2095.

The embodiments of the present invention as described hereinbefore provide an indented antimicrobial paper that may be used to provide a sanitized surface and/or prevent bacteria growth and/or microbe growth and/or migration thereof. It should be understood that the indented antimicrobial paper may be suitable for use on any surface which requires sanitary conditions or like applications. Additionally, the foregoing uses of the indented antimicrobial paper are an illustration of various uses of the indented antimicrobial paper. However, the foregoing uses of the indented antimicrobial paper should not be construed as limiting the present invention and other uses of the indented antimicrobial paper may be implemented by one skilled in the art.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

1. A paper for providing a sanitized surface, the paper comprising:
a base defined by a length and a width wherein the base has a top side and a bottom side wherein the bottom side is opposite the top side and further wherein the base forms a plane;
an antimicrobial surface associated with the top side of the base wherein the antimicrobial surface covers the top side; and
a plurality of depressions formed in the base wherein the plurality of depressions extend outside of the plane of the base.

2. The paper of claim 1 wherein the plurality of depressions are uniform across the base.

3. The paper of claim 1 wherein the plurality of depressions are continuous from side to side on the base.

4. The paper of claim 1 further comprising:
an antimicrobial layer associated with the bottom surface of the base.

5. The paper of claim 1 further comprising:
a paper layer associated with the top side of the base wherein the paper layer is located between the antimicrobial surface and the base.

6. The paper of claim 1 further comprising:
a water resistant layer associated with the top side of the base wherein the water resistant layer is located between the antimicrobial surface and the base.

7. The paper of claim 1 further comprising:
a water resistant layer associated with the bottom side of the base.

8. The paper of claim 1 further comprising:
a plurality of water resistant layers associated with the top side of the base wherein the plurality of water resistant layers is located between the base and the antimicrobial surface; and
a paper layer associated with the top side of the base wherein the paper layer is located between the antimicrobial surface and the base.

9. The paper of claim 1 further comprising:
a water resistant layer associated with the top side of the base wherein the water resistant layer is located between the base and the antimicrobial surface; and
a plurality of paper layers associated with the top side of the base wherein the plurality of paper layers is located between the antimicrobial surface and the base.

10. The paper of claim 1 further comprising:
a water resistant layer associated with the top side of the base wherein the water resistant layer is located between the antimicrobial surface and the base; and
a paper layer associated with the top side of the base wherein the paper layer is located between the antimicrobial surface and the base.

11. The paper of claim 1 further comprising:
a water resistant layer associated with the antimicrobial surface.

12. A process for making a paper, the process comprising the steps of:
providing a sheet having a first side and a second side wherein the second side is opposite the first side;
applying an antimicrobial layer to the first side of the sheet; and
forming an indentation in the sheet wherein the indentation is uniform across the sheet.

13. The process of claim 12 wherein the indentation is continuous along the sheet.

14. The process of claim 12 further comprising the step of:
applying a water resistant layer to the second side of the sheet.

15. The process of claim 12 further comprising the step of:
applying a water resistant layer to the first side of the sheet; and
scoring the water resistant layer wherein the antimicrobial layer is associated with the water resistant layer.

16. The process of claim 12 further comprising the step of:
applying a water resistant layer to the first side of the sheet;
scoring the water resistant layer; and
adhering a paper layer to the water resistant layer wherein the antimicrobial layer is associated with the paper layer.

17. The process of claim 12 further comprising the step of:
applying a plurality of water resistant layers to the first side of the sheet wherein the antimicrobial layer is associated with the plurality of water resistant layers.

18. The process of claim 12 further comprising the step of:
adhering a paper layer to the first side of the sheet wherein the antimicrobial layer is associated with the paper layer.

19. The process of claim 12 further comprising the step of:
applying a plurality of water resistant layers to the first side of the sheet; and
adhering a plurality of paper layers to the plurality of paper layers wherein the antimicrobial layer is associated with the plurality of water resistant layers.

20. The process of claim 12 further comprising the step of:
applying a water resistant layer to the antimicrobial layer.

21. The process of claim 12 further comprising the step of:
applying an antimicrobial surface to the second side of the sheet.

22. The process of claim 12 further comprising the step of:
adhering a paper layer to the first side of the sheet wherein the antimicrobial layer is associated with the paper layer.

23. The process of claim 12 further comprising the step of:
shredding the sheet.

24. The process of claim 12 further comprising the step of:
printing indicia onto the sheet.

25. The process of claim 12 further comprising the step of:
dividing the sheet into a plurality of sheets.

26. A method for using a paper to protect against contamination, the method comprising the steps of:
providing a sheet having a perimeter wherein the sheet has a bottom surface and a top surface wherein the top surface is opposite the bottom surface wherein an antimicrobial surface substantially covers the top surface and further wherein the sheet has an indented texture;
positioning the sheet on a surface wherein the bottom surface of the sheet is adjacent to the surface; and
positioning an object on the antimicrobial surface wherein the object is within the perimeter of the sheet.

27. The process of claim 26 further comprising the step of:
wrapping the antimicrobial surface around the object.

28. The process of claim 26 further comprising the step of:
enclosing the object within the sheet wherein the object is surrounded by the antimicrobial surface.

29. The process of claim 26 further comprising the step of:
separating a liquid from the object on the antimicrobial surface wherein the liquid is associated with the indented texture of the sheet.
